(19) United States
(12) Patent Application Publication
(10) Pub. No.: US 2014/0371591 A1
(43) Pub. Date: Dec. 18, 2014

(54) METHOD FOR AUTOMATICALLY DETECTING MID-SAGITTAL PLANE BY USING ULTRASOUND IMAGE AND APPARATUS THEREOF

(71) Applicant: SAMSUNG ELECTRONICS CO., LTD., Suwon-si (KR)

(72) Inventors: Hae-kyung Jung, Seoul (KR); Hee-chul Yoon, Seoul (KR); Hyun-taek Lee, Seoul (KR); Yong-je Kim, Yongin-si (KR); Jae-hyun Kim, Seoul (KR); Young-ho Moon, Suwon-si (KR)

(73) Assignee: SAMSUNG ELECTRONICS CO., LTD., Suwon-si (KR)

(21) Appl. No.: 14/368,130

(22) PCT Filed: Dec. 21, 2012
(86) PCT No.: PCT/KR2012/011247

§ 371 Date: Jun. 23, 2014

(30) Foreign Application Priority Data
Dec. 22, 2011 (KR) 10-2011-0140404

(51) Int. Cl. G06T 7/00; A61B 8/08; G06K 9/00

(52) U.S. Cl. CPC G06T 7/0012; G06K 9/00248; G06K 9/00255; A61B 8/0866; G06T 2207/10132; G06T 2207/30044; G06T 2207/30201; G06K 2209/05
USPC 600/443; 382/103

(57) ABSTRACT

Provided are a method and apparatus for automatically detecting a mid-sagittal plane of an object by using an ultrasonic image. The method includes obtaining an ultrasonic image for detecting the mid-sagittal plane; detecting a face of the object from the ultrasonic image; detecting a landmark from the detected face; detecting landmark information which includes at least one of brightness and a form of the detected landmark; and detecting a mid-sagittal plane of the object by using the landmark information.

### CROSS-REFERENCE TO RELATED APPLICATIONS

This application is a National Stage Entry of PCT/KR2012/011247, filed on Dec. 21, 2012, which claims priority to Korean patent application No. 10-2011-0140404, filed on Dec. 22, 2011 in the Korean Patent Office, the entire disclosures of which are herein incorporated by reference in their entirety.

### TECHNICAL FIELD

One or more exemplary embodiments relate to a method and apparatus for detecting and providing a mid-sagittal plane of an object automatically by using an ultrasonic image.

### BACKGROUND

Due to non-invasive and non-destructive characteristics, an ultrasonic system is widely used in the medical field to obtain internal information of an object. The ultrasonic system has a very important role in the medical field, as the ultrasonic system may provide high-resolution internal images of an object to doctors in real time, without having to perform a surgical operation in which the object may be incised and observed.

An ultrasonic image is utilized for an early diagnosis of any abnormality in chromosomes or the nervous system, such as Down syndrome of a fetus. In order to determine a location of the fetus through a visual examination performed by a doctor and measure accurate fetal biometrics, a fetus is diagnosed by obtaining an image of a mid-sagittal plane of a fetus and measuring a fetal crown rump length (CRL), nuchal translucency (NT), and intracranial translucency (IT) in the image of the mid-sagittal plane.

However, if a doctor manually searches for an image of a mid-sagittal plane of a fetus, the manual search may take a lot of time, and accuracy may vary according to a location or posture of the fetus, a skill level of a doctor, and the quality of an ultrasonic image. Thus, there are often cases of measuring fetal biometrics at a portion other than a mid-sagittal plane due to work efficiency, which causes clinical problems.

Therefore, it is necessary to provide a method and an apparatus for accurately detecting a mid-sagittal plane of a fetus.

### SUMMARY

One or more aspects of the exemplary embodiments may provide a method and apparatus for detecting and providing a mid-sagittal plane automatically by using an ultrasonic image.

The exemplary embodiments may provide a method and apparatus for detecting and providing a mid-sagittal plane of an object automatically by using an ultrasonic image.

According to an aspect of an exemplary embodiment, there may be provided a method of detecting a mid-sagittal plane of an object by using an ultrasonic image, the method including: obtaining an ultrasonic image for detecting the mid-sagittal plane; detecting a face of the object from the ultrasonic image; detecting a landmark from the detected face; detecting landmark information which includes at least one of brightness and a form of the detected landmark; and detecting a mid-sagittal plane of the object by using the landmark information.

The detecting of the landmark information may include detecting another landmark from the detected face based on distance information or angle information between the detected landmark and the other detected landmark.

The detecting of the mid-sagittal plane of the object may include: comparing a predetermined reference value to at least one of the brightness and the form of the detected landmark, and detecting the mid-sagittal plane of the object based on a difference between the predetermined reference value and the at least one of the brightness and the form of the detected landmark based on the comparing.

The method may further include: determining if a lighter detected landmark corresponds to the mid-sagittal plane, or a darker detected landmark which is darker than the lighter detected landmark corresponds to the mid-sagittal plane; based on a result of the determining, detecting a highest or lowest brightness value of the landmark; and defining the highest or lowest brightness value of the landmark as the predetermined reference value.

The method may further include: obtaining another ultrasonic image which corresponds to the mid-sagittal plane of the object; detecting other landmark information which comprises at least one of brightness and a form of another landmark from the other ultrasonic image; and defining the predetermined reference value by using information about the detected other landmark information.

The method may further include displaying a user interface (UI) which comprises at least one of whether an output ultrasonic image corresponds to the mid-sagittal plane and a probability by which the output ultrasonic image may correspond to the mid-sagittal plane.

The obtaining of the ultrasonic image may include obtaining an ultrasonic image for detecting mid-sagittal planes for a corresponding plurality of objects, and the detecting of the face of the object may include detecting faces of the objects which respectively correspond to the plurality of objects.

The method may further include outputting the landmark information which comprises at least one from among a location, a type, the brightness, the form, and a reference value of the detected landmark.

According to another aspect of an exemplary embodiment, there may be provided a method of detecting a mid-sagittal plane of an object, the method including: obtaining an ultrasonic image for detecting the mid-sagittal plane; detecting a face of the object from the ultrasonic image; detecting face form information of the object based on the detected face; comparing the face form information to a reference value; and based on a result of the comparing, detecting the mid-sagittal plane of the object based on a difference between the face form information and the reference value.

The method may further include: obtaining another ultrasonic image which corresponds to the mid-sagittal plane of the object; extracting face form information from the other ultrasonic image; and defining an average value of the extracted face form information as the reference value.

The method may further include outputting the face form information which comprises at least one from among a type, a shape, and a reference value of the detected face.

According to another aspect of an exemplary embodiment, there may be provided an ultrasonic image processing apparatus configured to detect a mid-sagittal plane of an object, the apparatus including: an image receiver configured to receive an ultrasonic image for detecting the mid-sagittal plane; a detection controller configured to detect a face of the object from the ultrasonic image, to detect a landmark from the detected face, to detect landmark information which comprises at least one of a location, brightness, a form and a reference value of the landmark, and to detect a mid-sagittal plane of the object by using the landmark information; and a landmark information storage configured to store the landmark information.

The detection controller may be configured to control the landmark information to be output by an external display.

The detection controller may include a face detector configured to detect the face of the object; a landmark detector configured to detect the landmark from the detected face and thus, to detect the landmark information; and a mid-sagittal detector configured to detect the mid-sagittal plane of the object from the ultrasonic image by using the landmark information.

The landmark information storage may be further configured to store the landmark information, and the landmark detector may be configured to detect another landmark based on at least one of distance information and angle information between the other landmark and the detected landmark.

Based on a result of comparing the reference value to at least one of the brightness and the form of the detected landmark information, the mid-sagittal detector may be configured to detect an ultrasonic image, which has a least difference between the reference value and the at least one of the brightness and the form, as the mid-sagittal plane of the object.

The landmark information storage may be configured to store additional landmark information which indicates whether a lighter detected landmark corresponds to the mid-sagittal plane, or a darker detected landmark which is darker than the lighter detected landmark corresponds to the mid-sagittal plane, and from among brightness values of the landmark detected from the ultrasonic image for detecting a mid-sagittal plane of the object, the mid-sagittal detector may be configured to define a highest or lowest brightness value of each of the landmarks as the reference value based on the landmark information.

The landmark detector may be configured to extract landmark information which includes at least one of brightness and a form of the landmark from the ultrasonic image which corresponds to the mid-sagittal plane of the object, obtain an average value of at least one of the brightness and the form of the landmark, and define the average value as a reference value of the landmark.

The image receiver may be configured to obtain an ultrasonic image for detecting mid-sagittal planes of a corresponding plurality of objects, and the face detector may be configured to detect a face of the object from the ultrasonic image which includes the plurality of objects.

The detection controller may be configured to control a user interface (UI), which includes at least one of whether an output ultrasonic image corresponds to the mid-sagittal plane and a probability by which the output ultrasonic image may correspond to the mid-sagittal plane, to be output by an external display unit.

According to another aspect of an exemplary embodiment, there may be provided an ultrasonic image processing apparatus configured to detect a mid-sagittal plane of an object, the apparatus including: an image receiver configured to receive at least one ultrasonic image for detecting the mid-sagittal plane from an external device; a face form information storage configured to store a reference value of a face form of the object; a detection controller configured to detect a face of the object from the ultrasonic image, detect form information of the detected face, set a window which comprises the face in order to detect the face form of the detected face and, based on a result of comparing the detected face form to the reference value, detect the mid-sagittal plane of the object based on a difference between the reference value and the detected face form.

The detection controller may be configured to control the face form information, which includes at least a type, a shape, and a reference value of the detected face form, to be output by an external display unit.

According to the exemplary embodiments, it is possible to automatically detect a mid-sagittal plane of an object by using a relatively-inexpensive basic 1D ultrasonic probe, as well as a mechanically-swept 1D probe or a 2D probe.

Furthermore, in order to detect a mid-sagittal plane of an object, at least one ultrasonic image is created and detected without having to create 3D volume data. Therefore, a mid-sagittal plane may be detected by using simple equipment and without requiring a complicated process.

Furthermore, a user input is only necessary for designating an area which is deemed to include a mid-sagittal plane. Accordingly, after at least one ultrasonic image is obtained, an additional input is not necessary, and thus, user input may be minimized.

Furthermore, in a case of detecting a landmark, a reference landmark is detected first, and then, a location of another landmark is detected by considering anatomical characteristics. Therefore, a more accurate mid-sagittal plane may be detected.

Furthermore, a face form and a landmark of an object may be detected entirely or partially through a training-based algorithm. A mid-sagittal plane may be detected by detecting a landmark or a face form of an object included in an ultrasonic image using a reference value obtained through the training-based algorithm.

### BRIEF DESCRIPTION OF THE DRAWINGS

FIG. 1 is a block diagram illustrating an ultrasonic system for automatically detecting a mid-sagittal plane by using an ultrasonic image, according to an exemplary embodiment;

FIG. 2 is a block diagram illustrating a structure of an ultrasonic data obtaining unit in the ultrasonic system for automatically detecting a mid-sagittal plane by using an ultrasonic image, according to an exemplary embodiment;

FIG. 3 is a block diagram illustrating a structure of an ultrasonic image processing apparatus in an ultrasonic system for automatically detecting a mid-sagittal plane by using an ultrasonic image, according to an exemplary embodiment;

FIG. 4 is a block diagram illustrating a structure of an ultrasonic image processing apparatus in an ultrasonic system for automatically detecting a mid-sagittal plane by using an ultrasonic image, according to another exemplary embodiment;

FIG. 5 is a block diagram illustrating a structure of an ultrasonic image processing apparatus in an ultrasonic system for automatically detecting a mid-sagittal plane by using an ultrasonic image, according to another exemplary embodiment;

FIG. 6 is a flowchart illustrating a method of detecting a mid-sagittal plane by using an ultrasonic image, according to an exemplary embodiment;

FIG. 7 is a flowchart illustrating a method of detecting a mid-sagittal plane by using a landmark, according to another exemplary embodiment;

FIG. 8 is a flowchart illustrating a method of detecting a mid-sagittal plane by using a brightness value of a landmark, according to another exemplary embodiment;

FIG. 9 is a flowchart illustrating a method of detecting a mid-sagittal plane by using a face form of an object, according to another exemplary embodiment;

FIG. 10 is a flowchart illustrating a method of defining a reference value by using a plurality of ultrasonic images which correspond to a mid-sagittal plane, according to exemplary embodiments;

FIGS. 11A and 11B illustrate an example of a screen for displaying a method of detecting a mid-sagittal plane by an ultrasonic image of an object, according to an exemplary embodiment; and

FIGS. 12A and 12B illustrate examples of a screen for displaying whether an output ultrasonic image corresponds to a mid-sagittal plane detected by using an ultrasonic image of an object, according to an exemplary embodiment.

### DETAILED DESCRIPTION OF THE EXEMPLARY EMBODIMENTS

The following description and accompanying drawings are provided for better understanding of the exemplary embodiments. In the following description, well-known functions or constructions are not described in detail if it is determined that they would obscure the exemplary embodiments due to unnecessary detail. Like numbers refer to like elements throughout the description of the figures.

All words or terms used herein have the same meaning as commonly understood by one of ordinary skill in the art to which the exemplary embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein. Detailed illustrative exemplary embodiments are disclosed herein. However, specific structural and functional details disclosed herein are merely representative for purposes of describing exemplary embodiments. The exemplary embodiments may, however, may be embodied in many alternate forms and should not be construed as limited to only the exemplary embodiments set forth herein.

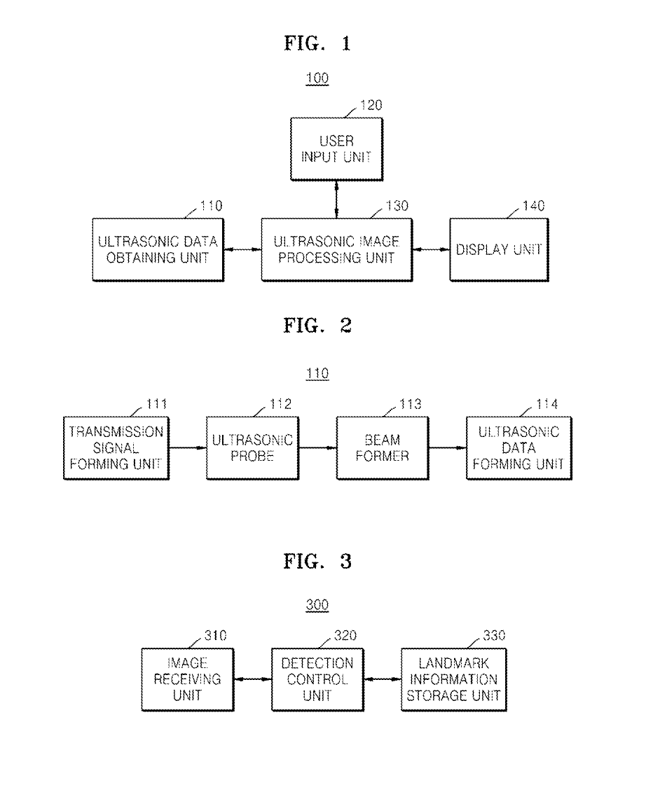

FIG. 1 is a block diagram illustrating an ultrasonic system 100 for automatically detecting a mid-sagittal plane by using an ultrasonic image, wherein the ultrasonic system 100 includes an ultrasonic image processing apparatus 130, according to an exemplary embodiment.

Referring to FIG. 1, the ultrasonic system 100, according to an exemplary embodiment, includes an ultrasonic data obtaining unit 110, a user input unit 120, the ultrasonic image processing apparatus 130, and a display unit 140.

The ultrasonic data obtaining unit 110 transmits an ultrasonic signal into an object and receives a return ultrasonic signal, which is reflected from the object, thus obtaining ultrasonic data which corresponds to each frame which is Pi (1≦i≦N). The return ultrasonic signal reflected from the object is referred to as an ultrasonic echo signal. For convenience of description, the object is described as a fetus, but it is not limited thereto.

FIG. 2 is a block diagram illustrating a structure of the ultrasonic data obtaining unit 110 in the ultrasonic system 100 for automatically detecting a mid-sagittal plane by using an ultrasonic image, according to an exemplary embodiment.

Referring to FIG. 2, the ultrasonic data obtaining unit 110 includes a transmission signal forming unit 111, an ultrasonic probe 112 which includes a plurality of transducer elements (not illustrated), a beam former 113, and an ultrasonic data forming unit 114.

The transmission signal forming unit 111 forms a transmission signal for obtaining each frame which is Pi (1≦i≦N) by considering a location and a convergence point of a transducer element.

When the transmission signal is transmitted from the transmission signal forming unit 111, the ultrasonic probe 112 converts the transmission signal into an ultrasonic signal and transmits the ultrasonic signal to the object, for example, a fetus. Then, the ultrasonic probe 112 receives an ultrasonic echo signal, which is reflected from the object, thus forming a reception signal.

A one-dimensional (1D) probe, a mechanically-swept 1D probe, a three-dimensional (3D) probe, or a two-dimensional (2D) array probe may be used as the ultrasonic probe 112.

When the reception signal is provided by the ultrasonic probe 112, the beam former 113 performs analog-to-digital conversion, thus forming a digital signal. The beam former 113 receives and converges a digital signal by considering a location and a convergence point of the transducer element, thus forming a reception convergence beam.

The ultrasonic data forming unit 114 forms ultrasonic data by using the reception convergence beam provided from the beam former 113. Additionally, the ultrasonic data forming unit 114 may also execute various signal processing for forming ultrasonic data, such as gain adjustment and filtering, on the reception convergence beam.

Referring back to FIG. 1, the user input unit 120 receives user input information. The input information may include input information for defining an area for obtaining an ultrasonic image for detecting a mid-sagittal plane. The user input unit 120 may include a control panel, a mouse, a keyboard, and the like.

The ultrasonic image processing apparatus 130 controls the ultrasonic data obtaining unit 110 to obtain an ultrasonic image in the defined area. When the ultrasonic data obtaining unit 110 receives at least one ultrasonic image, the ultrasonic image processing apparatus 130 detects a face of the object from the ultrasonic image and detects at least one landmark from the detected face, thus detecting landmark information, which includes at least one of brightness and a form of each landmark. Then, the ultrasonic image processing apparatus 130 detects a mid-sagittal plane of the object from at least one ultrasonic image by using the detected landmark information, and provides the detected mid-sagittal plane to the user.

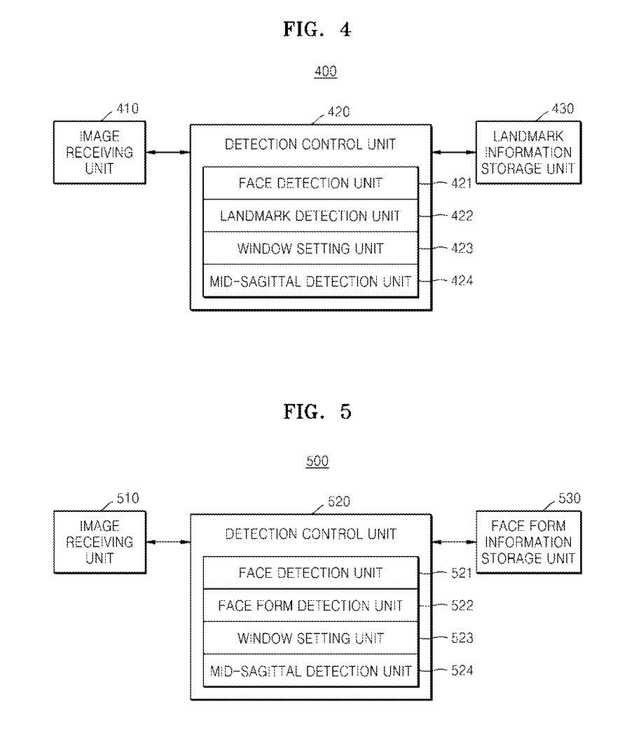

FIG. 3 is a block diagram illustrating a structure of an ultrasonic image processing apparatus 300 for detecting a mid-sagittal plane by using an ultrasonic image, according to an exemplary embodiment. The ultrasonic image processing apparatus 300 may correspond to the ultrasonic image processing apparatus 130 of FIG. 1, and may also correspond to ultrasonic image processing apparatuses 400 and 500, respectively shown in FIGS. 4 and 5 described later.

Referring to FIG. 3, the ultrasonic image processing apparatus 300 according to an exemplary embodiment may include an image receiving unit 310 (e.g., image receiver), a detection control unit 320 (e.g., detection controller), and a landmark information storage unit 330 (e.g., landmark information storage).

The image receiving unit 310 receives at least one ultrasonic image from a prediction area for detecting a mid-sagittal plane. The prediction area is an area for obtaining an ultrasonic image. The prediction area may be defined according to a user input signal. The image receiving unit 310 may externally receive an ultrasonic image. Otherwise, the image receiving unit 310 may include an ultrasonic photographing device (not illustrated), so as to autonomously obtain an ultrasonic image. The ultrasonic photographing device may be the ultrasonic data obtaining unit 110 described above.

When an ultrasonic image, obtained through the ultrasonic probe 112, is output through an external display device so that a user may view the ultrasonic image, the user may define a prediction area which may include a mid-sagittal plane visually by using the output ultrasonic image. Otherwise, the prediction area, which may include a mid-sagittal plane, may be defined by an external control signal.

Then, the image receiving unit 310 may request the ultrasonic data obtaining unit 110 to transmit an ultrasonic image of the defined area. Accordingly, the image receiving unit 310 may receive at least one ultrasonic image of the prediction area, which is an area defined for detecting the mid-sagittal plane and predicted to include the mid-sagittal plane, from the ultrasonic data obtaining unit 110 or an external device.

Additionally, when a 1D probe is used, a plurality of ultrasonic cross-sectional images may be obtained by photographing the defined area at certain distance intervals. When a 2D probe or a mechanically-swept 1D probe is used, an ultrasonic image is obtained, and then, divided into a plurality of ultrasonic cross-sectional images to detect the mid-sagittal plane.

The detection control unit 320 controls overall operations of the ultrasonic image processing apparatus 300. Basically, the detection control unit 320 runs based on operation programs stored in an internal storage device, so as to construct a basic platform environment for the ultrasonic image processing apparatus 300 and provide arbitrary functions by executing an application program according to user selection.

Specifically, the detection control unit 320 detects a face of the object in the ultrasonic image, detects at least one landmark in the detected face, and thus, detects landmark information which includes at least one of brightness and a form of each landmark. Then, the detection control unit 320 may detect a mid-sagittal plane of the object from at least one ultrasonic image by using the detected landmark information. Additionally, the detection control unit 320 may control the landmark information, which includes at least one from among a location, a type, brightness, a form, and a reference value of the detected landmark, to be output to the display unit 140.

The landmark information storage unit 330 may store landmark information including at least one from among reference values of a location, brightness, and a form, according to an exemplary embodiment. The landmark information storage unit 330 may include all types of storage media such as random-access memory (RAM), read-only memory (ROM), hard disk drive (HDD), flash memory, CD-ROMs, and digital versatile disc (DVD).

A reference value of brightness and a form of each landmark, stored in the landmark information storage unit 330, may be pre-defined. Additionally, the detection control unit 320 may detect landmark information, about at least one of brightness and a form of each landmark, from a plurality of ultrasonic images which correspond to the mid-sagittal plane of the object, and define an average value of the detected landmark information as a reference value. Then, when the mid-sagittal plane is detected from at least one ultrasonic image of the object, landmark information is detected for each landmark of the detected mid-sagittal plane. The detected landmark information is reflected in defining a reference value, and thus, the reference value of each landmark may be updated.

FIG. 4 is a block diagram illustrating a structure of an ultrasonic image processing apparatus 400 in an ultrasonic system for automatically detecting a mid-sagittal plane by using an ultrasonic image, according to another exemplary embodiment.

Referring to FIG. 4, the ultrasonic image processing apparatus 400 according to another exemplary embodiment may include an image receiving unit 410, a detection control unit 420, and a landmark information storage unit 430. The image receiving unit 410 and the landmark information storage unit 430 respectively correspond to the image receiving unit 310 and the landmark information storage unit 330 of FIG. 3, and thus, a description thereof is not provided again.

According to another exemplary embodiment, the detection control unit 420 may include a face detection unit 421 (e.g., face detector), a landmark detection unit 422 (e.g., landmark detector), and a mid-sagittal plane detection unit 424 (e.g., mid-sagittal plane detector).

The image receiving unit 410 receives an ultrasonic image of a defined area, and transmits the received ultrasonic image to the face detection unit 421.

The face detection unit 421 may detect a face of the object in at least one ultrasonic image which is received from the image receiving unit 410. Then, the face detection unit 421 detects a landmark included in the face, and by using the detected landmark, detects an ultrasonic image which corresponds to a mid-sagittal plane.

Additionally, if there are two or more objects, such as twin fetuses, the ultrasonic image may, for example include two or more faces, and thus, as each object has a mid-sagittal plane, faces may be detected for each object, and a mid-sagittal plane may be detected for each face. If there is a plurality of faces to be detected, the detection control unit 420 may control a user interface (UI) to be output to the display device 140, so as to select at least one object to detect a mid-sagittal plane.

The landmark detection unit 422 detects at least one landmark in the face detected by the face detection unit 421, and thus, detects landmark information, which includes at least one of brightness and a form of each landmark. A specific area, which is commonly included in the object, may be designated as a landmark. In a case of a fetus, a thalamus, a nasal bone tip, a palate bone, or a cheekbone may be designated as a landmark.

Additionally, the landmark detection unit 422 may detect at least one landmark in the detected face, and detect other landmarks, based on at least one of distance information and angle information between landmarks in reference to the detected landmark. For example, as a palate bone is most easily and correctly detected among landmarks, the palate bone is designated as a reference landmark, and detected first in an ultrasonic image which includes a face of an object. Then, based on distance information or angle information between landmarks in reference to a location of the palate bone, other landmarks such as a thalamus, a nasal bone tip, a palate bone, and a cheekbone may be detected. Accordingly, after a landmark, which is relatively easy to detect, is detected, at least another landmark may be detected based on the detected landmark, by considering the distance information or the angle information between landmarks in which anatomic characteristics of the object are reflected. Thus, an accuracy of the detected mid-sagittal plane may be increased.

Additionally, the landmark detection unit 422 may extract landmark information, which includes at least one of brightness and a form of the landmark, from at least one ultrasonic image which corresponds to the mid-sagittal plane of the object. Then, the landmark detection unit 422 may obtain an average value of the at least one of the brightness and the form and may define the average value as a reference value.

The landmark detection unit 422 may reflect an ultrasonic image which corresponds to the mid-sagittal plane and update a reference value of a landmark stored in the landmark information storage unit 430.

The mid-sagittal plane detection unit 424 may detect a mid-sagittal plane of an object from at least one ultrasonic image by using landmark information. A reference value of brightness or a form of a landmark is stored in the landmark information storage unit 430, and the reference value is compared to at least one of the brightness and the form which is detected from the ultrasonic image. Then, based on a result of the comparing, an ultrasonic image with a least difference between the reference value and the at least one of the brightness and the form may be detected as the mid-sagittal plane.

A specific portion of which characteristics are changed, according to whether the specific portion is the mid-sagittal plane, is designated as a landmark. By newly defining a reference value for each detection result of the mid-sagittal plane based on the characteristics, the mid-sagittal plane may be detected by the mid-sagittal plane detection unit 424. For example, a thalamus and a cheekbone may appear darkest and a nasal bone tip and a palate bone may appear lightest in the ultrasonic image which corresponds to the mid-sagittal plane. On the contrary, the thalamus and the cheekbone appear darker, and the nasal bone tip and the palate bone appear lighter in the ultrasonic image, when located farther from the mid-sagittal plane.

Accordingly, landmark information, which includes whether a lighter landmark corresponds to the mid-sagittal plane or a darker landmark corresponds to the mid-sagittal plane for each landmark, may be further stored in the landmark information storage unit 430, and used for defining a reference value for each landmark and detecting a landmark. The mid-sagittal plane detection unit 424 may define a highest or lowest brightness value of each landmark, among brightness values of a landmark detected from at least one ultrasonic image which is obtained from an area predicted to include the mid-sagittal plane of the object, as a reference value of the brightness of a landmark. After defining the reference value, the mid-sagittal plane detection unit 424 may determine a difference between the reference value and the brightness value of the landmark detected from at least one ultrasonic image, and detect the mid-sagittal plane from an ultrasonic image with a least difference.

Landmark information may further include form information about the landmark, and the form information of the landmark, which is detected from an ultrasonic image, may be compared to a reference value stored in the landmark information storage unit 430. The form information of the landmark may include information about at least one of sharpness, a shape, and a size of an outline of the landmark. By further comparing the reference value to a value of a form of the landmark in addition to the brightness value of the landmark, an accuracy of detection of the mid-sagittal plane may be increased.

Information about the detected landmark, which includes a form, brightness, a location, a type such as a palate bone or a nasal bone tip, a reference value, information about a reference landmark such as whether the detected landmark is the reference landmark, or information about a distance or an angle between the detected landmark and the reference landmark, may be output via the display unit 140. Thus, information about the detected landmark may be provided to a user.

The detection control unit 420, according to another exemplary embodiment, may further include a window setting unit 423.

The window setting unit 423 sets a window which includes a landmark. The window may be set to include a location of the landmark included in a face in at least one ultrasonic image of an object.

In order to obtain another landmark after obtaining the reference landmark, the window may be set to cover a specific portion of a face which is deemed to include the reference landmark, so as to figure out a location of the reference landmark. Accordingly, if a palate bone is designated as a reference landmark, the reference landmark is located at a front center of a face of a fetus. Thus, a window may be set for an area which is deemed to include a reference landmark in the face of the fetus, in order to figure out a location of the reference landmark. If a location of another landmark is determined by using stored distance and angle information based on the reference landmark, a window may be set to include a location of each landmark for detecting a mid-sagittal plane and thus, information about each landmark may be detected.

The landmark detection unit 422 may detect landmark information which includes at least one of brightness and form of a landmark, which is included in the window set by the window setting unit 423. Additionally, the mid-sagittal plane detection unit 424 may detect a mid-sagittal plane by comparing the detected landmark information to a pre-defined reference value for each landmark, and then, selecting an ultrasonic image which has a highest possibility of corresponding to the mid-sagittal plane.

More specifically, the mid-sagittal plane detection unit 424 may detect landmark information which includes at least one of brightness or a form of a landmark, included in the window set by the window setting unit 423. Then, the mid-sagittal plane detection unit 424 may compare the detected value to a reference value of each landmark and, based on a result of the comparison, select an ultrasonic image with a least difference therebetween as an ultrasonic image which corresponds to the mid-sagittal plane.

FIG. 5 is a block diagram illustrating a structure of an ultrasonic image processing apparatus 500 in an ultrasonic system for automatically detecting a mid-sagittal plane by using an ultrasonic image, according to another exemplary embodiment. Referring to FIG. 5, the ultrasonic image processing apparatus 500, according to another exemplary embodiment, may include an image receiving unit 510 (e.g., image receiver), a detection control unit 520 (e.g., detection controller), and a face form information storage unit 530 (e.g., face form information storage). The image receiving unit 510 corresponds to the image receiving units 310 and 410 respectively shown in FIGS. 3 and 4, and thus, the description of the corresponding element will not be provided again here.

The detection control unit 520 may include a face detection unit 521, a face form detection unit 522, and a mid-sagittal plane detection unit 524, and may further include a window setting unit 523. The face detection unit 521, the window setting unit 523, and the mid-sagittal plane detection unit 524 that may be included in the detection control unit 520 correspond to elements which are included in the image processing apparatus 400, according to another exemplary embodiment. Thus, a description thereof is not provided again.

The face form detection unit 522 may detect a face form of an object from at least one ultrasonic image of the object, and thus, detect a mid-sagittal plane from at least one ultrasonic image which has a least difference between face form information and a reference value.

The face form information storage unit 530 may store a reference value of a face form. The face form detection unit 522 may detect a mid-sagittal plane by comparing the reference value of the face form to a face form which is detected from at least one ultrasonic image.

Based on a result of comparing a face form detected by the face form detection unit 522 to the reference value stored in the face form information storage unit 530, the mid-sagittal plane detection unit 524 may detect a mid-sagittal plane of the object from an ultrasonic image with a least difference between the face form and the reference value. Additionally, the detection control unit 520 may further include the window setting unit 523 to set a window to include a face of the object and to detect face form information in the set window.

The face form information for detecting a mid-sagittal plane may include information about a size, a shape, and sharpness of a face of the object. Through comparison of the face form information to a reference value, the mid-sagittal plane may be detected from an ultrasonic image.

The face form information, which includes information about a size, a shape, and sharpness of a face of the object, may be output via the display unit 140. Thus, information about a detected face form may be provided to a user.

The ultrasonic image processing apparatus 500, according to another exemplary embodiment, transmits a request to the ultrasonic data obtaining unit 110 and receives at least one ultrasonic image in a prediction area for detecting a mid-sagittal plane. Then, the ultrasonic image processing apparatus 500 detects a face of the object in the ultrasonic image, detects form information about the detected face, and sets a window to include the face. Then, the ultrasonic image processing apparatus 500 compares the form of the detected face to a reference value. Based on a result of the comparing, the ultrasonic image processing apparatus 500 may detect a mid-sagittal plane of the object from an ultrasonic image which has a least difference from the reference value. Unlike previous exemplary embodiments in which a landmark included in a face is used, in the current exemplary embodiment, a mid-sagittal plane may be detected from an ultrasonic image by using a face form.

Furthermore, the ultrasonic image processing apparatuses 400 and 500, according to exemplary embodiments, may both include the landmark detection unit 422 and the face form detection unit 522. More specifically, the ultrasonic image processing apparatuses 400 and 500, according to exemplary embodiments, may detect a face of an object, and then detect face form information as well as a landmark included in the detected face. Thus, by comparing the landmark and the face form to reference values thereof, the ultrasonic image processing apparatuses 400 and 500 may detect a mid-sagittal plane from an ultrasonic image with a least difference in the comparison of the landmark and the face form to reference values.

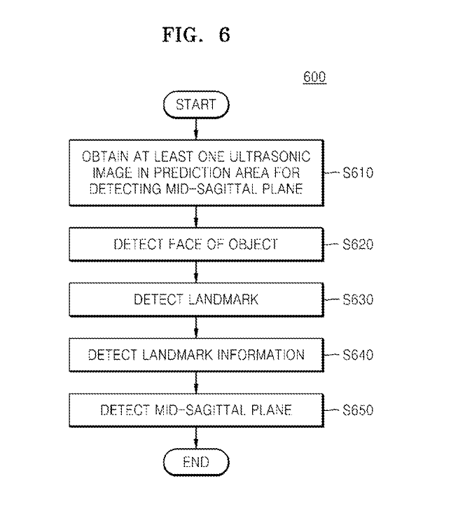

FIG. 6 is a flowchart illustrating a method 600 of detecting a mid-sagittal plane by using an ultrasonic image, according to an exemplary embodiment.

Referring to FIG. 6, in operation S610, at least one ultrasonic image is obtained from a prediction area for detecting a mid-sagittal plane. The prediction area may be defined according to a user input signal or an external control signal. An ultrasonic image for detecting the mid-sagittal plane of the object may be obtained from the prediction area.

In operation S620, a face of the object is detected from the obtained ultrasonic image. Then, in operation S630, at least one landmark included in the face is detected from the detected face. In operation S640, landmark information, which includes at least one of brightness and a form of the detected landmark, is detected. By using the detected landmark information, a mid-sagittal plane of the object may be detected from at least one ultrasonic image.

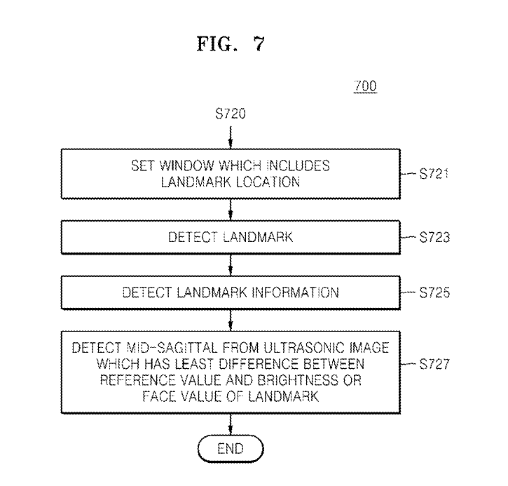

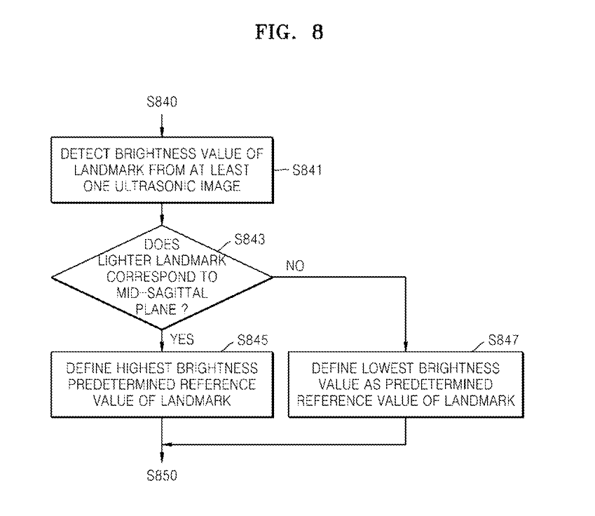

FIG. 7 is a flowchart illustrating a method 700 of detecting a mid-sagittal plane by using a landmark, according to another exemplary embodiment.

Operation S720 of FIG. 7 corresponds to operation S620 of FIG. 6. Therefore, the method 700 of detecting a mid-sagittal plane, before operation S721, may include operations S610 and S620.

Referring to FIG. 7, when a face of the object is detected from the obtained ultrasonic image in operation S720, a window, which includes a location of a landmark, is set based on the detected face, in operation S721. In operation S723, at least one landmark is detected in the set window, and in operation S725, information about the detected landmark is detected. If the landmark is detected by using a reference landmark, the reference landmark may be detected by setting a window to include a location of the reference landmark.

In operation S727, a predetermined reference value and at least one of brightness and a form of the detected landmark information are compared, and based on a result of the comparison, a mid-sagittal plane of the object may be detected from an ultrasonic image with a least difference between at least one of the brightness and the form, and the reference value.

FIG. 8 is a flowchart illustrating a method of detecting a mid-sagittal plane by using a brightness value of a landmark, according to another exemplary embodiment.

Operation S840 of FIG. 8 may correspond to operation S640 of FIG. 6. Therefore, the method of detecting a mid-sagittal plane, before operation S841, may include operations S610 through S640. Referring to FIG. 8, in operation S840, information about a detected landmark is detected. Then, in operation S843, it is determined if a lighter detected landmark is a mid-sagittal plane, or a darker detected landmark is a mid-sagittal plane. Characteristics information about a brightness value of each landmark in the mid-sagittal plane is stored for each landmark in the landmark information storage unit 430 and employed in operation S843.

As a result of the determination in S843, a highest value or a lowest value of the brightness value of the landmark is detected from at least one ultrasonic image. In operation S845, if the brightness value of the landmark is highest in the mid-sagittal plane, the highest value of the corresponding landmark in the obtained ultrasonic image is detected and defined as a reference value. On the contrary, in operation S847, if the brightness value of the landmark is lowest in the mid-sagittal plane, the lowest value of the corresponding landmark in the obtained ultrasonic image is detected and defined as a reference value.

When a reference value is defined for each landmark, in operation S850, a difference between the reference value and the brightness value of the detected landmark for each ultrasonic image may be calculated, and a mid-sagittal plane may be detected from an ultrasonic image with a least difference thereof. In a case of considering two or more landmarks, it may be determined if an ultrasonic image corresponds to the mid-sagittal plane based on an addition of differences between brightness values and the reference value. Operation S850 may correspond to operation S650 of FIG. 6.

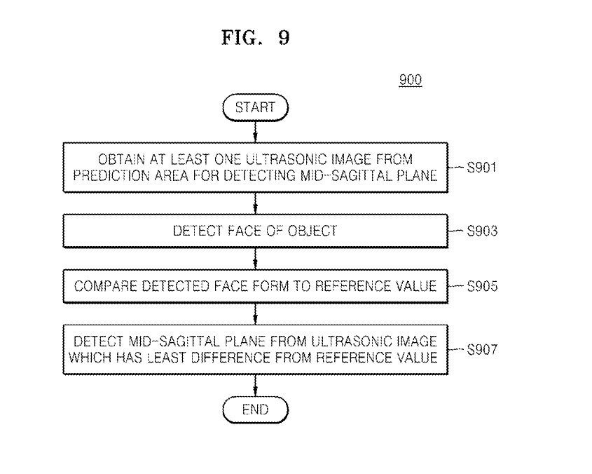

FIG. 9 is a flowchart illustrating a method 900 of detecting a mid-sagittal plane by using a face form of an object, according to another exemplary embodiment.

In operation S901, at least one ultrasonic image is obtained in a prediction area for detecting a mid-sagittal plane. Then, in operation S903, a face of an object is detected from the ultrasonic image. When the face is detected, in operation S905, face form information is detected and compared to a reference value. Based on a result of the comparison in operation S905, a mid-sagittal plane of the object may be detected from an ultrasonic image with a least difference between the face form information and the reference value, in operation S907.

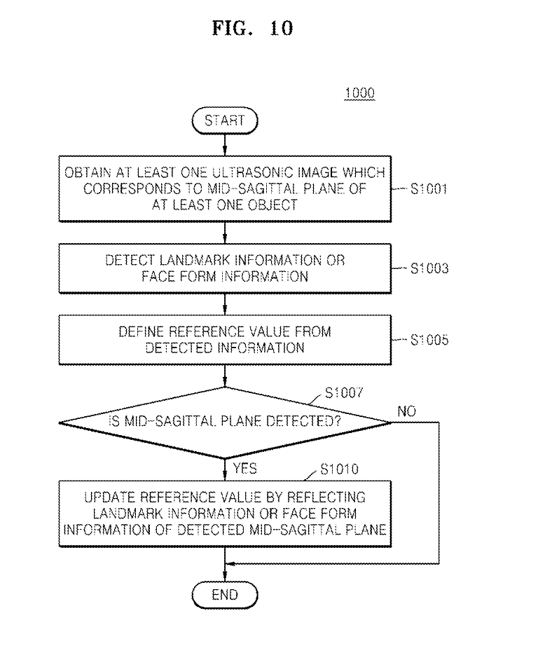

FIG. 10 is a flowchart illustrating a method 1000 of defining a reference value by using a plurality of ultrasonic images which correspond to a mid-sagittal plane, according to exemplary embodiments.

A face form of an object and a landmark may be detected partially or entirely through a training-based algorithm. That is, a face form and a landmark of an object may be detected by applying a plurality of ultrasonic images to the training-based algorithm, so as to obtain a reference value of the face form or the landmark of the object. Therefore, the face form or the landmark of the object may be detected by using a reference value obtained from the training-based algorithm, and thus, the mid-sagittal plane may be detected.

In operation S1001, the ultrasonic image processing apparatus 400 and 500 obtain at least one ultrasonic image, which corresponds to the mid-sagittal plane of an object. An ultrasonic image, which corresponds to the mid-sagittal plane obtained in operation S1001, may be obtained from an external device according to a control signal, or may be an ultrasonic image which corresponds to a mid-sagittal plane detected according to an exemplary embodiment and is stored in an external storage device. The obtained ultrasonic image is used to define a reference value of a landmark or a face form included in the object, and thus, may desirably be an ultrasonic image that corresponds to a mid-sagittal plane of the same kind of object.

In operation S1003, at least one of landmark information and face form information is detected from the ultrasonic image obtained in operation S1001. In operation S1005, a reference value may be defined based on the detected landmark information and face form information.

In operation S1005, the reference value of the landmark may be defined for each landmark and based on the landmark information and the face form information, which are detected from at least one ultrasonic image. Additionally, an average value of the landmark information and the face form information may be defined as a reference value. The average value may include an arithmetic mean value, a geometric mean value, and a harmonic mean value.

If the reference value is defined by using an ultrasonic image which corresponds to at least one mid-sagittal plane, and in operation S1007, the mid-sagittal plane is detected according to an exemplary embodiment, the reference value may be updated by reflecting landmark information or face form information about the detected mid-sagittal plane, in operation S1010. The reference value may be updated by reflecting the landmark information, if the mid-sagittal plane is detected according to an exemplary embodiment shown in FIG. 3 or 4, or by reflecting the face form information, if the mid-sagittal plane is detected according to an exemplary embodiment shown in FIG. 5.

There are two methods of defining a reference value for detecting a mid-sagittal plane by using a landmark, as shown in FIGS. 8 and 10. Accordingly, the detection control unit 420 according to an exemplary embodiment may control a UI to be output by the display unit 140 so that a user may set a method of defining a reference value for detecting a mid-sagittal plane.

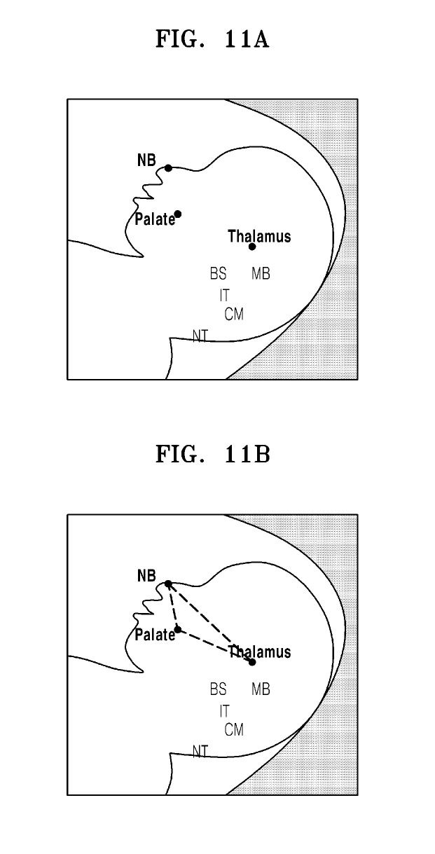

FIGS. 11A and 11B illustrate examples of a screen for displaying a method of detecting a mid-sagittal plane by an ultrasonic image of an object, according to an exemplary embodiment.

FIG. 11A shows an ultrasonic image of a fetus, in which landmarks such as a thalamus, a nasal bone tip, a palate bone, and a cheekbone may be identified.

FIG. 11B shows a window which is set to include landmarks to be considered to detect the mid-sagittal plane. Referring to FIG. 11B, a window, set to include a thalamus, a nasal bone tip, a palate bone, and a cheekbone, may be identified.

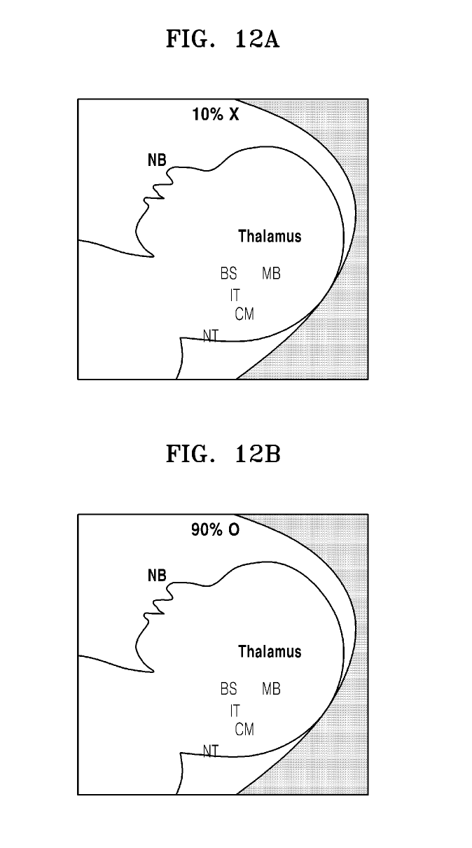

FIGS. 12A and 12B illustrate examples of a screen for displaying whether an output ultrasonic image corresponds to a mid-sagittal plane which is detected by using an ultrasonic image of an object, according to an exemplary embodiment.

Referring to FIGS. 12A and 12B, an ultrasonic image, which corresponds to a mid-sagittal plane, is detected, and an output ultrasonic image may be compared to the ultrasonic image, which corresponds to the mid-sagittal plane. Then, based on a result of the comparison, it may be determined if the output ultrasonic image corresponds to the mid-sagittal plane, according to a degree of correspondence to the mid-sagittal plane, and a result of the determination may be displayed. For example, if the degree of correspondence is less than or equal to 80%, the output ultrasonic image may be defined as not matching the mid-sagittal plane. If the degree of correspondence is more than 80%, the output ultrasonic image may be defined as matching the mid-sagittal plane.

In FIGS. 12A and 12B, a sign is used to display whether the ultrasonic image shows the mid-sagittal plane or not, but a display method is not limited thereto. Additionally, the degree by which the output ultrasonic image corresponds to the mid-sagittal plane may, for example, also be output as a probability.

The exemplary embodiments can be embodied as computer-readable codes on a computer-readable recording medium (including all devices with a data processing capability). The computer-readable recording medium is any data storage device that can store data which can be thereafter read by a computer system. Examples of the computer-readable recording medium include read-only memory (ROM), random-access memory (RAM), CD-ROMs, magnetic tapes, floppy disks, optical data storage devices, etc.

While the exemplary embodiments have been particularly shown and described with reference to certain exemplary embodiments thereof, it will be understood by those skilled in the art that various deletions, substitutions, and changes in form and details of the apparatus and method, described above, may be made therein without departing from the spirit and scope of the exemplary embodiments as defined by the appended claims. Therefore, the scope of the exemplary embodiments is defined not by the detailed description of the exemplary embodiments but by the appended claims, and all differences within the scope will be construed as being included in the exemplary embodiments.

1. A method of detecting a mid-sagittal plane of an object by using an ultrasonic image, the method comprising:
obtaining an ultrasonic image for detecting the mid-sagittal plane;
detecting a face of the object from the ultrasonic image;
detecting a landmark from the detected face;
detecting landmark information which comprises at least one of brightness and a form of the detected landmark; and
detecting a mid-sagittal plane of the object by using the landmark information.

2. The method of claim 1, wherein the detecting of the landmark information comprises:
detecting another landmark from the detected face based on distance information or angle information between the detected landmark and the other detected landmark.

3. The method of claim 1, wherein the detecting of the mid-sagittal plane of the object comprises:
comparing a predetermined reference value to at least one of the brightness and the form of the detected landmark, and
detecting the mid-sagittal plane of the object based on a difference between the predetermined reference value and the at least one of the brightness and the form of the detected landmark based on the comparing.

4. The method of claim 3, further comprising:
determining if a lighter detected landmark corresponds to the mid-sagittal plane, or a darker detected landmark which is darker than the lighter detected landmark corresponds to the mid-sagittal plane;
based on a result of the determining, detecting a highest or lowest brightness value of the landmark; and
defining the highest or lowest brightness value of the landmark as the predetermined reference value.

5. The method of claim 3, further comprising:
obtaining another ultrasonic image which corresponds to the mid-sagittal plane of the object;
detecting other landmark information which comprises at least one of brightness and a form of another landmark from the other ultrasonic image; and
defining the predetermined reference value by using information about the detected other landmark information.

6. The method of claim 1, further comprising displaying a user interface (UI) which comprises at least one of whether an output ultrasonic image corresponds to the mid-sagittal plane and a probability by which the output ultrasonic image may correspond to the mid-sagittal plane.

7. The method of claim 1, wherein the obtaining of the ultrasonic image comprises obtaining an ultrasonic image for detecting mid-sagittal planes for a corresponding plurality of objects, and
wherein the detecting of the face of the object comprises detecting faces of the objects which respectively correspond to the plurality of objects.

8. The method of claim 1, further comprising outputting the landmark information which comprises at least one from among a location, a type, the brightness, the form, and a reference value of the detected landmark.

9. A method of detecting a mid-sagittal plane of an object, the method comprising:
obtaining an ultrasonic image for detecting the mid-sagittal plane;
detecting a face of the object from the ultrasonic image;
detecting face form information of the object based on the detected face;
comparing the face form information to a reference value; and
based on a result of the comparing, detecting the mid-sagittal plane of the object based on a difference between the face form information and the reference value.

10. The method of claim 9, further comprising:
obtaining another ultrasonic image which corresponds to the mid-sagittal plane of the object;
extracting face form information from the other ultrasonic image; and
defining an average value of the extracted face form information as the reference value.

11. The method of claim 9, further comprising outputting the face form information which comprises at least one from among a type, a shape, and a reference value of the detected face.

12. An ultrasonic image processing apparatus configured to detect a mid-sagittal plane of an object, the apparatus comprising:
an image receiver configured to receive at an ultrasonic image for detecting the mid-sagittal plane;
a detection controller configured to detect a face of the object from the ultrasonic image, to detect a landmark from the detected face, to detect landmark information which comprises at least one of a location, brightness, a form and a reference value of the landmark, and to detect a mid-sagittal plane of the object by using the landmark information; and
a landmark information storage configured to store the landmark information.

13. The ultrasonic image processing apparatus of claim 12, wherein the detection controller is configured to control the landmark information to be output by an external display.

14. The ultrasonic image processing apparatus of claim 12, wherein the detection controller comprises:
a face detector configured to detect the face of the object;
a landmark detector configured to detect the landmark from the detected face and thus, to detect the landmark information; and
a mid-sagittal detector configured to detect the mid-sagittal plane of the object from the ultrasonic image by using the landmark information.

15. The ultrasonic image processing apparatus of claim 14, wherein the landmark information storage is further configured to store the landmark information, and
wherein the landmark detector is configured to detect another landmark based on at least one of distance information and angle information between the other landmark and the detected landmark.

16. The ultrasonic image processing apparatus of claim 14, wherein, based on a result of comparing the reference value to at least one of the brightness and the form of the detected landmark information, the mid-sagittal detector is configured to detect an ultrasonic image, which has a least difference between the reference value and the at least one of the brightness and the form, as the mid-sagittal plane of the object.

17. The ultrasonic image processing apparatus of claim 14, wherein the landmark information storage is configured to store additional landmark information which indicates whether a lighter detected landmark corresponds to the mid-sagittal plane, or a darker detected landmark which is darker than the lighter detected landmark corresponds to the mid-sagittal plane, and
wherein, from among brightness values of the landmark detected from the ultrasonic image for detecting a mid-sagittal plane of the object, the mid-sagittal detector is configured to define a highest or lowest brightness value of each of the landmarks as the reference value based on the landmark information.

18. The ultrasonic image processing apparatus of claim 14, wherein the landmark detector is configured to extract landmark information which comprises at least one of brightness and a form of the landmark from the ultrasonic image which corresponds to the mid-sagittal plane of the object, obtain an average value of at least one of the brightness and the form of the landmark, and define the average value as a reference value of the landmark.

19. The ultrasonic image processing apparatus of claim 14, wherein the image receiver is configured to obtain an ultrasonic image for detecting mid-sagittal planes of a corresponding plurality of objects, and
wherein the face detector is configured to detect a face of the object from the ultrasonic image which comprises the plurality of objects.

20. The ultrasonic image processing apparatus of claim 12, wherein the detection controller is configured to control a user interface (UI), which comprises at least one of whether an output ultrasonic image corresponds to the mid-sagittal plane and a probability by which the output ultrasonic image may correspond to the mid-sagittal plane, to be output by an external display unit.

21. An ultrasonic image processing apparatus configured to detect a mid-sagittal plane of an object, the apparatus comprising:
an image receiver configured to receive at least one ultrasonic image for detecting the mid-sagittal plane from an external device;
a face form information storage configured to store a reference value of a face form of the object;
a detection controller configured to detect a face of the object from the ultrasonic image, detect form information of the detected face, set a window which comprises the face in order to detect the face form of the detected face and, based on a result of comparing the detected face form to the reference value, detect the mid-sagittal plane of the object based on a difference between the reference value and the detected face form.

22. The ultrasonic image processing apparatus of claim 20, wherein the detection controller is configured to control the face form information, which comprises at least a type, a shape, and a reference value of the detected face form, to be output by an external display unit.

23. An ultrasonic apparatus, comprising:
an ultrasonic probe configured to obtain an ultrasound of an object;
a landmark detector configured to detect a visual landmark displayed in the ultrasound, the visual landmark being associated with the object; and
a detection controller configured to detect a mid-sagittal plane of the object based on the visual landmark.

24. The ultrasonic apparatus of claim 23, wherein the object comprises a fetus, and the visual landmark indicates one of thalamus, a nasal bone tip, a palate bone, and a cheekbone of the fetus.

25. The ultrasonic apparatus of claim 24, wherein the detection controller detects the mid-sagittal plane by comparing information related to the landmark with a reference value.
